(19) United States
(12) Patent Application Publication
(10) Pub. No.: US 2004/0258727 A1
(43) Pub. Date: Dec. 23, 2004

(54) Ophthalmic biomaterials and preparation thereof

(76) Inventors: Lina Liu, Hamilton (CA); Heather D. Sheardown, Nobleton (CA)

Correspondence Address: Ralph A. Dowell of DOWELL & DOWELL, P.C., Suite 309, 1215 Jefferson Davis Highway, Arlington, VA, 22202, US

(21) Appl. No.: 10/855,955

(22) Filed: May 28, 2004

(30) Foreign Application Priority Data
May 28, 2003 (CA) 2,430,185

(51) Int. Cl. C08G 65/48; C08L 71/12; A61F 2/00

(52) U.S. Cl. 424/423; 525/393

(57) ABSTRACT

Composite interpenetrating network (IPN) of PDMS and PNIPAAM was formed to generate polymers with oxygen and glucose permeability as well as improved wettability compared to PDMS homopolymers and greater mechanical strength than PNIPAAM homopolymers. Transparent vinyl and hydroxyl terminated PDMS/PNIPAAM IPNs (PDMS-V and PDMS-OH IPNs respectively) were successfully synthesized. Transmission electron microscopy images verified the structure of the IPNs. Surface analysis suggested that PNIPAAM was present on the surface as well as in the bulk material. PDMS-OH IPNs generated from a PDMS-OH matrix cured in the presence of solvent had the highest glucose permeability at 10⁻⁷ cm²/s, comparable to that of the native cornea. The LCST phenomenon remained in these materials, although changes were not as abrupt as with pure PNIPAAM. These results suggest that these materials may be further developed as ophthalmic biomaterials or for controlled drug release applications.

### CROSS REFERENCE TO RELATED APPLICATIONS

This patent application claims the priority benefit from Canadian Patent Application Serial No. 2,430,185 filed on May 28, 2003 entitled OPHTHALMIC BIOMATERIALS AND PREPARATION THEREOF.

### FIELD OF THE INVENTION

This invention relates to novel biomaterial compositions, particularly to use of these materials for ophthalmic membranes, such as corneas; and to methods of preparing the biometric compositions.

### BACKGROUND OF THE INVENTION

The multiplicity of physical and chemical properties, such as, for example, transparency, mechanical strength, oxygen permeability, permeability to glucose and other nutrient molecules as well as cellular compatibility [1, 11], required of a biomaterial in order to be used as the basis for an artificial cornea, makes selection of a suitable material challenging. For many corneal blind patients, synthetic based artificial corneas offer the best hope for replacement of the diseased tissue and restoration of sight. The synthetic materials must have a number of properties, including transparency and the ability to maintain appropriate corneal cells. Related to latter property is the necessity for the material to be able to transmit both oxygen and small molecular weight nutrients such as glucose to the inner structures of the eye.

Polydimethylsiloxane (PDMS) based elastomers have been used inter alia in biomedical ophthalmic applications [1, 30], such as in contact lens [2, 3, 4] and artificial corneas [5, 6, 7] due to their high oxygen permeability, transparency and good mechanical properties. However, particularly for use in artificial cornea, permeability to glucose has been suggested to be an important determinant to success. Therefore, while the oxygen permeability of these materials is adequate for good ophthalmic health, the lack of glucose permeability limits their potential for such use. On the other hand, ophthalmic biomaterials hydrogels, such as, for example, poly (hydroxyethyl methacrylate) (PHEMA) and N-vinyl pyrrolidone (NVP), which possess good glucose permeability, however, lack the necessary oxygen permeability and mechanical strength for long term application.

Poly (N-isopropyl acrylamide) (PNIPAAM), a hydrogel that has been extensively studied as an intelligent polymeric matrix for use in biotechnology and bioengineering [8], shows a reversible phase transition at its lower critical solution temperature. This transition temperature may be altered by copolymerization with another hydrophilic monomer such as acrylamide [9, 10]. However, a serious limitation to the widespread use of PNIPAAM hydrogels in many applications is the low mechanical strength of the gels in a highly swollen state, although PNIPAAM has been used in ophthalmic and other drug delivery applications with good results [9, 11].

Interpenetrating polymer networks (IPN's) have been described as an intimate entanglement of two crosslinked networks [12] and consisting of two or more network polymers, with at least one having been polymerized and/or crosslinked in the immediate presence of others [13]. The interlocked structures of the crosslinked components are believed to ensure the stability of the bulk and surface morphology. While there are many reports in the literature of silicone hydrogel composites [e.g. 14, 15] and hydrogel grafted silicones [16], interpenetrating polymer networks composed of silicones and hydrogels, including PHEMA [17, 18,19], poly vinyl alcohol (PVA) [20, 21] and poly (methacrylic acid) (PMAA) [22, 23] have only recently been developed. In order to maintain the high glucose permeability of the hydrogel phase, a high level of interconnectivity, large hydrogel domains and surface connectivity of the hydrogel phase is necessary. While Turner and Cheng [22, 23] examined some of these parameters, other work has focused on preparation methods. Although PNIPAAM IPN's have been widely studied, the majority are synthesized using a combination of PNIPAAM with a second relatively hydrophilic component [24, 25, 26, 27, 28].

### SUMMARY OF THE INVENTION

It is an object of the present invention to provide a novel matrix composition of at least two polymers having improved physical and chemical properties for use as a biomaterial.

It is a further object of the present invention to provide processes for the preparation of said matrix composition.

Accordingly, the invention provides in one aspect a method of preparing a biomaterial matrix composition as hereinabove defined, said method comprising polymerizing a pre-polymer precursor of one of said first polymer or said second polymer in the presence of the other of said first or second polymers.

More particularly, in this aspect of the invention, there is provided a process for the manufacture of an interpenetrating polymer network biomaterial matrix composition comprising at least a first hydrophilic polymer material and a second hydrophobic polymer material in intimate entanglement one with the other; said process comprising:

a) vulcanising to effect cross-linking of a vulcanisable first hydrophobic polymer backbone precursor of the general formula (I)

T-QN-T (I)

wherein n is greater than or equal to O;

Q is an internal siloxane group of the formula (II)

—O—Si(R¹R²)— (II)

wherein R¹, R² may be the same or different and selected from the group consisting of H, provided that both R¹, R² are not hydrogen on the same internal silicon atom; alkoxy, alkyl, aryl, functional aryl, a crosslinked organic group linking to another silicone-based chain, or a group having an internal siloxane group of the formula III:

(OSiR³R⁴)ₘR⁵ (III)

wherein m is 24 0; and R³, R⁴ and R⁵ for each internal siloxane group may be the same or different selected from the group consisting of alkoxy, siloxy, alkyl, functional alkyl, aryl, functional aryl, independently H, with the proviso that not more than one of R³, R⁴ and R⁵ is H on the same silicon atom; or a crosslinked organic group linking to another silicone-based chain;

T is a radical of the formula (IV);

—OSiR⁶R⁷R⁸ (IV)

wherein R⁶ R⁷ R⁸ may be the same or different and selected from the group consisting of H with the proviso that the silicon atom has no more than one H; alkoxy, siloxy, alkyl, functional alkyl, aryl, functional aryl, or a crosslinked organic group linking to another silicone-based chain;

in a suitable first solvent with a suitable cross-linking agent, to produce a cross-linked elastomer;

b) removing said solvent to form a film of said elastomer;

c) adding a cross-linkable hydrogel compound in a suitable second solvent to said elastomeric film to effect swelling of said elastomer film and form a swollen admixture;

d) reacting said hydrogel compound in said admixture with a suitable cross-linking agent to produce cross-linked hydrogel in said admixture;

and removing said second solvent to produce said interpenetrating polymer network biomaterial matrix composition;

the improvement wherein said first silicone polymer backbone precursor concentration in said first solvent is not greater than 60% W/V in said vulcanization step.

Preferably, the precursor concentration is selected from between 5-30% W/V.

A preferred silicone polymer backbone precursor is a hydroxyl terminated polydimethyl siloxane (PDMS) having an approximate M.W. of 60,000 and a viscosity of approximately 5,000 centistokes.

Preferably, the hydrogel is a poly N-isopropyl anoglamide (PNIPAAM).

Thus, suitable subsituents for R¹ to R⁸ include linear, branched and cyclic saturated alkyl groups having up to 20 carbons such as, for example, methyl, ethyl, n-propyl, i-propyl, n-butyl, i-butyl, t-butyl, pentyl, n-hexyl, cyclohexyl, linear, branched and cyclic groups, alkoxy groups having up to 20 carbon atoms, such as methoxy, ethoxy, propoxy, butoxy, cyclopentyloxy; unsaturated derivatives of fatty acids having up to 20 carbon atoms, such as linolenyl groups; unsaturated cyclic hydrocarbon groups, such as cyclopentadienyl; and aryl groups, such as phenyl, tolyl, benzyl and naphthyl. These substituents may be substituted at a substitutable position with a halogen such as fluorine, chlorine, bromine or iodine, or with a hydroxy, alkoxy, or amino group. It will be appreciated, however, that the substituents should not materially affect the hydrophobic properties of the silicone polymer backbone.

The silicone polymer of formula (1) has a molecular weight (g/mol) of between about 500 and about 1,000,000 and preferably between about 2000 and 100,000. The compounds of formula (1) include those polysiloxanes having a “linear” backbone, as well as those having a “branched” backbone structure.

Linkers to other silicone-based polymer chains may be based on silicones or organic residues and are formed by reactions familiar to those skilled in the art. Preferably, the linkers are selected from O atoms or complex functional groups, for example, functional alkyl and functional aryl groups.

The suitable substituents may also include linkages to other silicone chains formed by crosslinking processes as noted above, and may include linkages such as R¹ to R⁸═OSiXYZ, where X, Y, Z are taken form the same groups as T,Q; (CH₂) and pSiXYZ, where p=1-10.

The methods according to the invention as hereinabove defined provides for the synthesis of network matrix compositions which have a high level of surface connectivity, large hydrogel domains, and favourable glucose and oxygen permeability with acceptable physical properties for use as ophthalmic biomaterials.

Accordingly, in a further aspect, the invention provides an interpenetrating polymer network biomaterial matrix composition when made by a process as defined hereinabove.

Preferably, the biomaterial matrix composition is in the form of a membrane, preferably an ophthalmic membrane for use as an artificial cornea or lens.

In a further aspect, the invention provides an interpenetrating polymer matrix biomaterial composition comprising at least a first polymer and a second polymer in intimate entanglement one with the other, when made by a process as defined hereinabove.

Accordingly, in one aspect, the invention provides an interpenetrating polymer network biomaterial matrix composition comprising at least a first polymer and a second polymer in intimate entanglement one with the other, the first polymer being a poly dialkylsiloxane such as poly diethylsiloxane, ethylhydeosiloxane, methylhydrosiloxane or their block copolymers and the second polymer being a poly N-alkyl acrylamide including the polymers of acrylamide (H₂C═CHCONH₂) and N-cyclopropylacrylamide N-n-propylacrylamide, N-ethylacrylamide, N-tert-butylacrylamideor N,N-dialkyl-substituted polyacrylamide such as N,N-dimethylacrylamide, N,N-fiethylacrylamide. The matrices may be prepared using other hydrogels prepared using other hydrophilic polymers such as, polymers of acrylic acid (H₂C═CHCOOH), methacrylic acid (H₂C═C(CH₃)COOH), 1-vinyl-2pyrrolidinone, poly(2-hydroxyethyl methacrylate)(CH₂═CHCOOCH₂CH₂OH), ethylene oxide, methacrylic acid, glycidyl methacrylate and their copolymers. These hydrophilic polymers can be used to prepare the PDMS hydrogel with adjusted LCST or without LCST depending on different biological applications. The friction of hydrophilic polymer which are associated with PDMS, the water uptake and others properties varies for different hydrogel species.

The methods according to the invention as hereinabove defined provides the synthesis of PDMS-PNIPAAM network matrix compositions which have a high level of surface connectivity, sufficiently large hydrogel domains for transport of materials while maintaining transparency and the characterization of these polymers for use as ophthalmic biomaterials.

### BRIEF DESCRIPTION OF THE DRAWINGS

In order that the invention may be better understood, preferred embodiments will now be described by way of example only, with reference to the accompanying drawings, wherein.

FIGS. 1a and 1b are Fourier Transform Infrared Spectroscopy (FTIR) charts of PDMS and PNIPAAM, respectively;

FIG. 1c is the FTIR chart of vinyl terminated PDMS PNIPAAM networks;

FIGS. 1d and 1e are the FTIR charts of 36.3 wt % and 22.6 wt % hydroxyl terminated PDMS PNIPAAM networks respectively;

FIGS. 2 and 3 are differential scanning calorimetry graphs of the networks produced according to the invention;

FIG. 4 is a graph of the effect of copolymerization on the LCST of the PDMS PNIPAAM networks, according to the invention;

FIGS. 5a and 5b are bar charts showing some mechanical properties of the biomaterial network composition, according to the invention;

FIG. 6 is a graph of the effect of a cross-linking agent on PNIPAAM content in a composition according to the invention;

FIG. 7 is a graph showing the effect of the content of PNIPAAM in a composition on water uptake according to the invention;

FIG. 8 is a bar chart of water uptake relative to the amount of PDMS in a curing solution in the vulcanization step according to the invention;

FIG. 9 is a bar chart of water contact angles at the surfaces of various compositions according to the invention;

FIGS. 10a, 10b and 10c represents an atomic force microscopy (AFM) image for pure vinyl- and hydroxy-terminated PDMS host polymers; and for network compositions according to the invention;

FIGS. 11a and 11b are bar charts representing surface roughness analysis on vinyl terminated PDMS PNIPAAM networks and hydroxyl terminated PDMS PNIPAAM networks membranes respectively according to the invention;

FIG. 12 represents an AFM phase image of a hydroxyl terminated PDMS PNIPAAM network polymer according to the invention;

FIG. 13 is a graph of the change in glucose permeability with permeation temperature above the lower critical solution temperature.

FIGS. 14a and 14b are bar charts of transparency of vinyl and hydroxyl terminated PDMS PNIPAAM networks according to the invention; and

FIGS. 15a, 15b and 15c represent TEM images for pure PDMS (A) and hydroxyl and vinyl terminated PDMS PNIPAAM networks according to the invention.

### DETAILED DESCRIPTION OF REFERRED EMBODIMENTS

In this specification, the following terms have the meaning defined, unless otherwise described:

Hydrophobic defines groups or molecules that would not normally be soluble in water;

Hydrophilic defines groups or molecules that would normally be soluble in water;

Alkyl means an aliphatic hydrocarbon which may be linear, branched, cyclic or alkenyl having up to 20 carbon atoms;

Aryl means a hydrocarbon residue base, having up to 20 carbons and containing at least one conjugated cyclic structure, which cyclic structure may contain an 0 or N, and which cyclic structure may be substituted at a substitutable position with an alkyl group;

Alkoxy means OR, where R is alkyl, functional alkyl, aryl or functional aryl;

Siloxy means OSiR⁹R¹⁰R¹¹ wherein R⁹R¹⁰ and R¹¹ may be the same or different and selected from alkyl, functional alkyl, aryl or functional aryl groups, alkoxy, or other siloxy groups OH, or suitable other radicals as hereinabove defined.

The present invention provides a process for the manufacture of an interpenetrating polymer network biomaterial matrix composition comprising at least a first hydrophobic polymer material and a second hydrophilic polymer material in intimate entanglement one with the other. The process includes

a) vulcanising to effect cross-linking of a vulcanisable first silicone polymer backbone precursor of the general formula (I)

T-Qₙ-T (I)

wherein n is greater than or equal to O;

Q is an internal siloxane group of the formula (II)

—O—Si(R¹R²)— (II)

wherein R¹, R² may be the same or different and selected from the group consisting of H, provided that both R¹, R² are not hydrogen on the same internal silicon atom; alkoxy, alkyl, aryl, functional aryl, a crosslinked organic group linking to another silicone-based chain, or a group having an internal siloxane group of the formula III:

(OSiR³R⁴)ₘR⁵ (III)

wherein m is ≧0; and R³, R⁴ and R⁵ for each internal siloxane group may be the same or different selected from the group consisting of alkoxy, siloxy, alkyl, functional alkyl, aryl, functional aryl, independently H, with the proviso that not more than one of R³, R⁴ and R⁵ is H on the same silicon atom; or a crosslinked organic group linking to another silicone-based chain;

T is a radical of the formula (IV);

—OSiR⁶R⁷R⁸ (IV)

wherein R⁶ R⁷ R⁸ may be the same or different and selected from the group consisting of H with the proviso that the silicon atom has no more than one H; alkoxy, siloxy, alkyl, functional alkyl, aryl, functional aryl, or a crosslinked organic group linking to another silicone-based chain;

in a suitable first solvent with a suitable cross-linking agent, to produce a cross-linked elastomer;

b) removing said solvent to form a film of said elastomer;

c) adding a cross-linkable hydrogel compound in a suitable second solvent to said elastomeric film to effect swelling of said elastomer film and form a swollen admixture;

d) reacting said hydrogel compound in said admixture with a suitable cross-linking agent to produce cross-linked hydrogel in said admixture;

and removing said second solvent to produce said interpenetrating polymer network biomaterial matrix composition;

the improvement wherein said first silicone polymer backbone precursor concentration in said first solvent is not greater than 60% W/V in said vulcanization step.

Preferably, the precursor concentration is selected from between 5-30% W/V.

A preferred silicone polymer backbone precursor is a hydroxyl terminated polydimethyl siloxane (PDMS) having an approximate M.W. of 60,000 and a viscosity of approximately 5,000 centistokes.

Preferably, the hydrogel is a poly N-isopropyl anoglamide (PNIPAAM).

Thus, suitable subsituents for R¹ to R⁸ include linear, branched and cyclic saturated alkyl groups having up to 20 carbons such as, for example, methyl, ethyl, n-propyl, i-propyl, n-butyl, i-butyl, t-butyl, pentyl, n-hexyl, cyclohexyl, linear, branched and cyclic groups, alkoxy groups having up to 20 carbon atoms, such as methoxy, ethoxy, propoxy, butoxy, cyclopentyloxy; unsaturated derivatives of fatty acids having up to 20 carbon atoms, such as linolenyl groups; unsaturated cyclic hydrocarbon groups, such as cyclopentadienyl; and aryl groups, such as phenyl, tolyl, benzyl and naphthyl. These substituents may be substituted at a substitutable position with a halogen such as fluorine, chlorine, bromine or iodine, or with a hydroxy, alkoxy, or amino group. It will be appreciated, however, that the substituents should not materially affect the hydrophobic properties of the silicone polymer backbone.

The silicone polymer of formula (1) has a molecular weight (g/mol) of between about 500 and about 1,000,000 and preferably between about 2000 and 100,000. The compounds of formula (1) include those polysiloxanes having a “linear” backbone, as well as those having a “branched” backbone structure.

Linkers to other silicone-based polymer chains may be based on silicones or organic residues and are formed by reactions familiar to those skilled in the art. Preferably, the linkers are selected from O atoms or complex functional groups, for example, functional alkyl and functional aryl groups.

The suitable substituents may also include linkages to other silicone chains formed by crosslinking processes as noted above, and may include linkages such as R¹ to R⁸═OSiXYZ, where X, Y, Z are taken form the same groups as T,Q; (CH₂) and pSiXYZ, where p=1-10.

Those skilled in the art will recognize there are several different methods by which silicone polymers may be crosslinked into elastomers, including, for example, hydrosilylation catalyzed by metals or radicals, room temperature moisture cure and high temperature free radical cure. Other crosslinking processes are described for example, in Silicon in Organic, Organometallic and Polymer Chemistry, Brook, M A, Wiley, 2000, Chap. 9.

### EXAMPLE

The following description provides general procedures exemplified with references to PDMS and PNIPAAM-polymers.

Materials and Methods

Vinyl terminated (5000 cst, 48,000 MW) poly (dimethylsiloxane) (PDMS) kits (Sylgard 184™) were purchased from Dow Corning Chemical Co. Hydroxyl terminated (2000 cst, 35,000 MW) PDMS prepolymers were obtained from Sigma Aldrich. The low viscosity of these prepolymers eliminated the degassing step and resulted in the formation of more transparent polymer films. The vinyl terminated PDMS films were prepared as directed by the manufacturer. Briefly, the resin, and curing agent were mixed in a 10:1 ratio and poured into a glass petri dish. The films were cured at room temperature 24 hours, or at 65° C. for four hours and 100° C. for one hour. The hydroxyl terminated PDMS films were prepared by mixing the prepolymer, a crosslinker (tetraorthosilicate, TEOS) and a tin (II)-2-ethylhexanoate catalyst in a 100:10:3 ratio. Furthermore, to increase the amount of PNIPAAM in the IPN, PDMS films were prepared in solvent. Briefly, the catalyst, prepolymer and crosslinker were mixed with toluene. Solvent to polymer ratios were varied to determine the effect of this parameter on the networks formed from no solvent to an 87:13, solvent:polymer mixture. The polymer/solvent mixtures were poured onto water and allowed to cure at room temperature (approximately 3-5 days). Unreacted monomer was extracted from the films using hexane. The PDMS films were dried completely and weighed prior to network formation.

Matrix Network Formation

N-isopropyl acrylamide (NIPMM) monomer was purified by recrystallization in n-hexane. The monomer (30% w/w based on solvent), crosslinker (bisacrylamide, 3% mol/mol, crosslinker: monomer) and the UV sensitive initiator (xanthone, 2%, w/w, initiator: monomer) were added to tetrahyde furan solvent. Other initiators and crosslinking agents were also examined. The monomer mixture was allowed to swell the PDMS film for a period of 4 hours. The swollen PDMS-NIPAAM films were degassed at room temperature and placed 1.5˜4.0 cm from a UV lamp, having an intensity of 8 W and a wavelength of 312 nm for 12 hours. The films were removed from the lamps and reaction continued for a period of 4 hours. Unreacted NIPAAM monomer was extracted from the films using THF. The films were dried and weighed to determine the approximate PNIPAAM content. Depending the swelling time and the PDMS film used as the host, films containing between 0 and 45%, defined as:

$$PNIPAAM ( \% ) = \frac{{m}_{PDMS} - {m}_{network}}{{m}_{PDMS}} \times 100 \%$$

(wt %) PNIPAAM were prepared.

Matrices were also prepared using poly (2-hydroxyethyl methacrylate) (PHEMA) and poly (N-vinyl pyrrolidone) (PVP) as the guest polymers. The LCST phenomenon of the PNIPAAM containing interpenetrating networks was altered or removed by copolymerization of the NIPAAM during matrix formation with varying amounts of acrylamide (AAm) or acrylic acid (AAc).

Characterization of the PDMS/PNIPAAM Matrix Networks Bulk Characterization

Fourier Transform Infrared Spectroscopy (FTIR) was used to confirm the presence of the two polymers in the network. Water swelling studies were performed for the various membranes using Milli-Q water. Equilibrium swollen mass and dry mass were used to determine the water uptake, defined as:

$$Water Uptake = \frac{{m}_{swollen} - {m}_{dry}}{{m}_{dry}} \times 100 \%$$

for each of the membranes prepared. The transition temperatures of the samples were determined using a TA instruments 2910 differential scanning calorimeter (DSC) over the temperature range −20° to 200° C. at a heating rate of 15° C./min for Tg determination and 0−60° C. at a heating rate of 1 °C./min for LCST determination. Stress-strain measurements were carried out using an Instron tensile tester with a load range of 50 N and a crosshead speed of 50 mm/minute at room temperature. Samples were examined in both the dry and the swollen state. The domain size of the PNIPAAM domains was examined using laser scanning confocal microscopy of the water swollen samples. A fluorescent marker (FITC) was used to distinguish the PNIPAAM domains from the rubbery PDMS support. The domain size of the PNIPAAM domains was examined using Transmission Electron Microscopy (TEM) and Scanning Electron Microscopy (SEM). The transparency of the various network samples was assessed qualitatively by confocal microscopy. Transmission electron microscopy measurements of the samples were also made to assess the bulk morphology.

Surface Characterization

Sessile drop advancing and receding water contact angles were determined for the various network surfaces including the PDMS control. Milli Q water was used with a drop volume of approximately 0.02 mL. X-ray Photoelectron Spectroscopy (XPS) was performed at the Surface Interface Ontario, University of Toronto. Low resolution and high resolution C1s spectra were obtained for the various samples at takeoff angles of 90° and 20°. Atomic Force Microscopy (AFM) was performed at the Brockhouse Institute for Materials Research at McMaster University using a Digital Instruments Nanoscope III. Scan sizes of 10 μm×10 μm were used for each sample.

Glucose and Oxygen Permeation Measurements

Glucose permeation studies were carried out at room temperature and at 37° C. using standard two compartment diffusion cells. For glucose permeation measurements, the donor chamber was filled with the permeant dissolved in phosphate buffered saline at pH 7.4 and the receptor chamber was filled with the same buffer containing no permeant. The receptor chamber was sampled at specified time intervals and the volume removed replaced with fresh buffer. An approximate infinite sink condition was maintained in all experiments. The samples were analyzed for glucose using a glucose assay kit (Sigma Chemical Co., St. Louis Mo.), using a Beckman DU-640 spectrophotometer. To determine the permeability of the membranes to oxygen, a similar method was used. Oxygen was bubbled continuously through the donor solution. Oxygen was initially removed from the receptor solution by degassing. The oxygen concentration on the receptor side was monitored continuously using a make, model oxygen probe.

Results and Discussion

PDMS-PNIPAAM Network Matrix Synthesis

Glucose, as a model small molecule, permeability of the networks was expected to be highly dependent on the hydrogel content and also on the size and continuity of the PNIPAAM domains. Therefore, to optimize the hydrogel content of the networks and to control the domain size, fabrication parameters including functionality of the PDMS host, the PDMS curing method, the effect of the addition of solvent during the hydrogel curing step and the effect of crosslinker and initiator concentrations were examined. The hydrogel content of the polymers was relatively unaffected by the concentration of either the crosslinker or the initiator. However, curing the PDMS host in a solvent was found to result in significant changes in greater porosity, swellability, and to allow for the incorporation of larger amounts of PNIPAAM during the subsequent IPN formation. Solvent curing was possible with PDMS of different terminal functionality; curing rates with the vinyl terminated PDMS prepolymers were extremely slow. Much of the testing was performed with hydroxyl terminated PDMS networks.

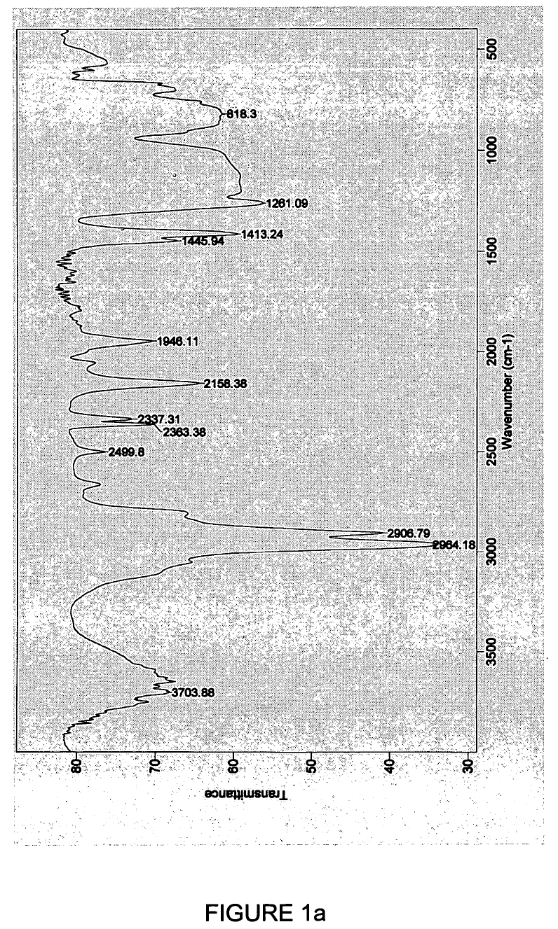

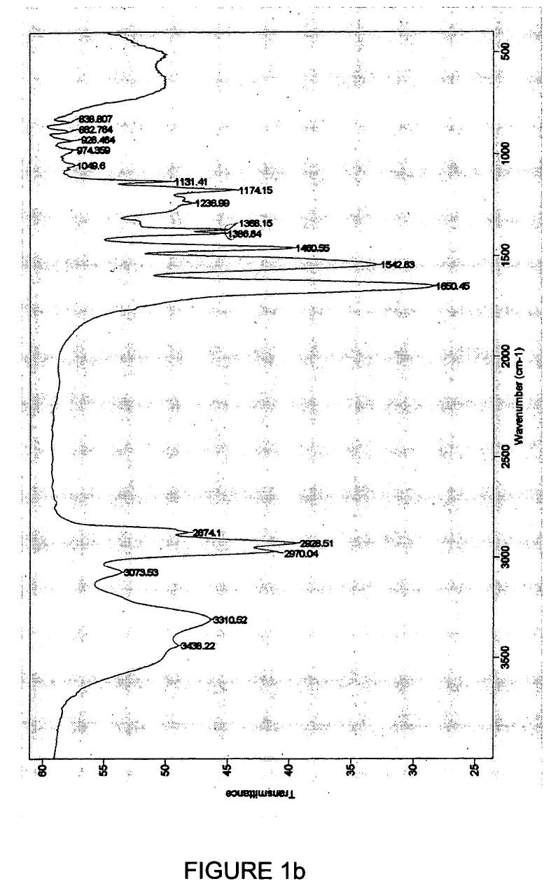

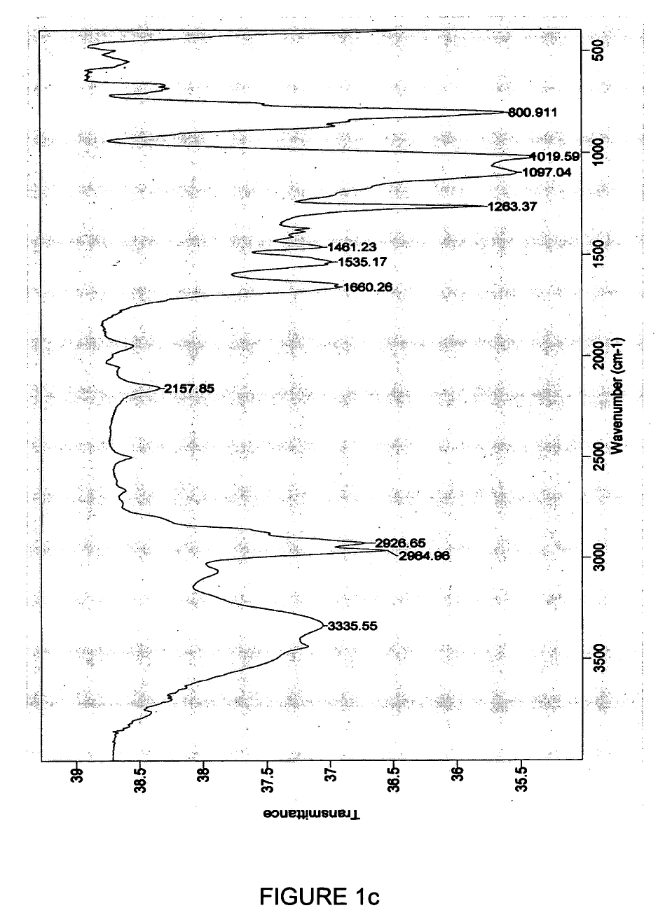

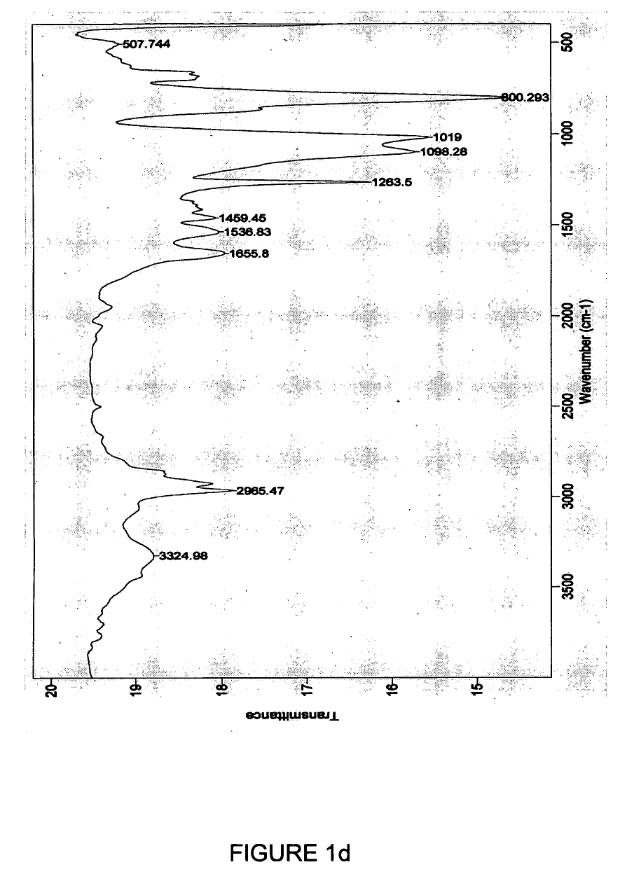

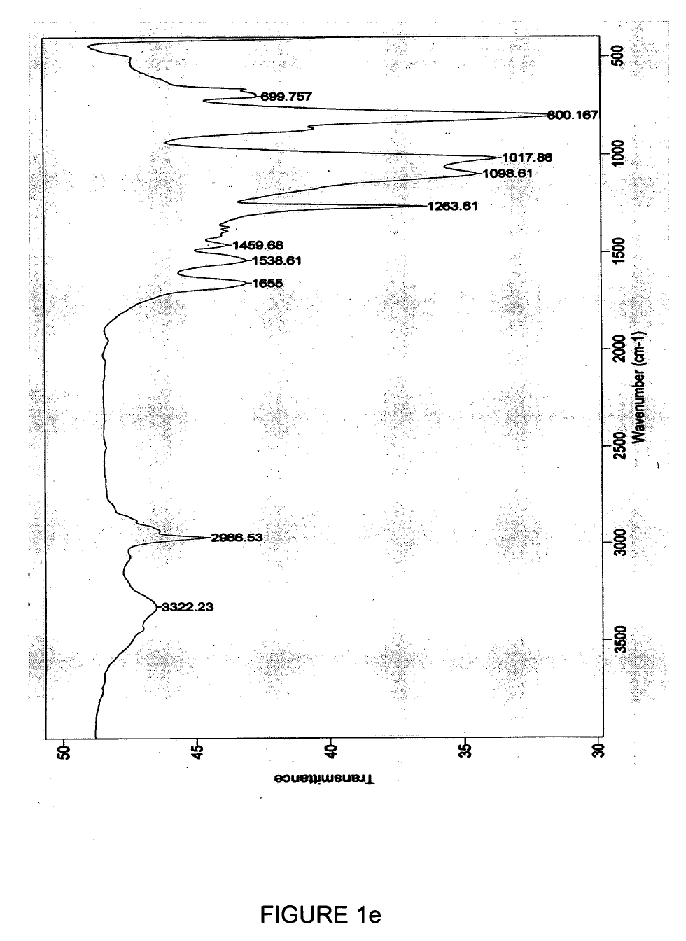

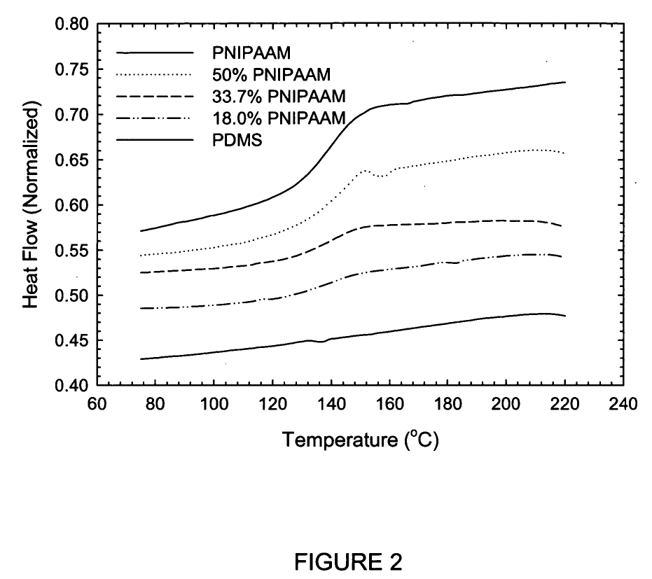

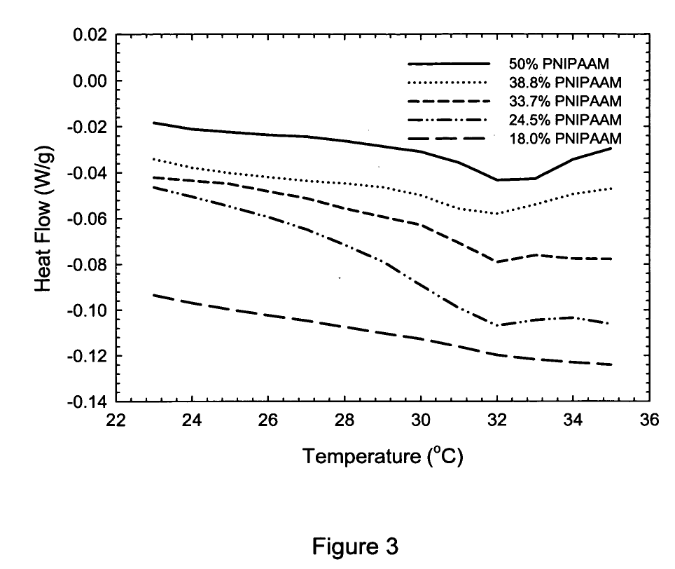

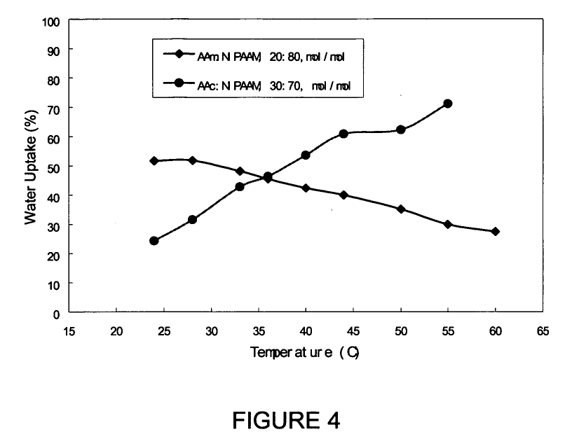

FIGS. 1a and 1b are Fourier Transform Infrared Spectroscopy (FTIR) charts of PDMS and PNIPAAM, respectively. FIG. 1c is the FTIR chart of vinyl terminated PDMS PNIPAAM networks, and FIGS. 1d and 1e are the FTIR charts of 36.3 wt % and 22.6 wt % hydroxyl terminated PDMS PNIPAAM networks respectively. The FTIR analyses, shown in FIGS. 1a-1e demonstrate that both PDMS and PNIPAAM are present in the networks formed. Specifically, peaks at ˜1000, 1450 and 2970 cm⁻¹ are characteristic of the PDMS while peaks at 1370, 1450, 1640, 2970 and 3310 cm⁻¹ provide evidence for PNIPAAM incorporation. Clear increases in the PNIPAAM peaks as a function of the measured PNIPAAM content were observed. DSC (Differential scanning calorimety) analyses provide further evidence of network formation, as shown in FIGS. 2 and 3. In FIG. 2, the appearance of a glass transition peak in the PNIPAAM networks is clearly noted. The LCST of the PNIPAAM guest polymer is shifted only slightly by network formation as shown in FIG. 3, indicating that copolymers of NIPMM and PDMS are not being formed during network formation. In all cases, the observed transition peaks become more distinct with increasing hydrogel content of the networks. However, as shown in Table A and FIG. 4, copolymerization of the NIPAAM with either acrylic acid or acrylamide during network formation either elevated or eliminated this transition temperature. More particularly, FIG. 4 shows the effect of copolymerization of the NIPAAM with acrylic acid or acrylamide on LCST of PDMS hydrogel. It is shown that the LCST of the network polymers are eliminated or weakened.

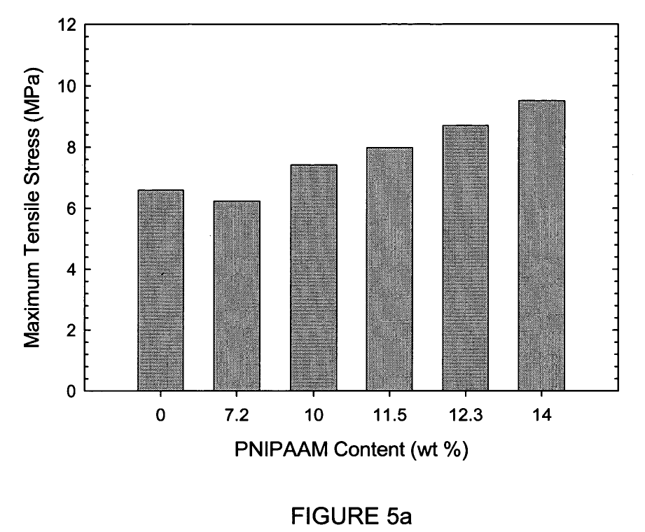

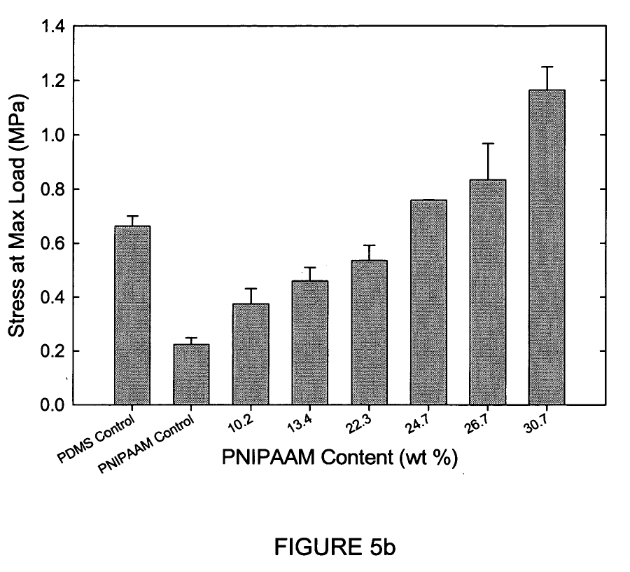

The mechanical properties of the network polymers, summarized in FIGS. 5a and 5b, were somewhat surprising. The vinyl terminated PDMS-PNIPAAM networks all showed an increased stress at maximum load relative to the PDMS control, despite the much lower value for the PNIPAAM control under wet conditions. While the tensile strength of the hydroxyl terminated PDMS control was approximately an order of magnitude lower than that measured for the vinyl control polymer, increases in the stress at maximum load with increasing PNIPAAM content were also found in these polymers. Furthermore, somewhat surprisingly, the mechanical properties of these polymers in the swollen state were only 10-20% lower than the polymers in the dry state. It is believed that these improved mechanical properties result from the presence of the rigid PNIPAAM entrapped within a continuous PDMS phase.

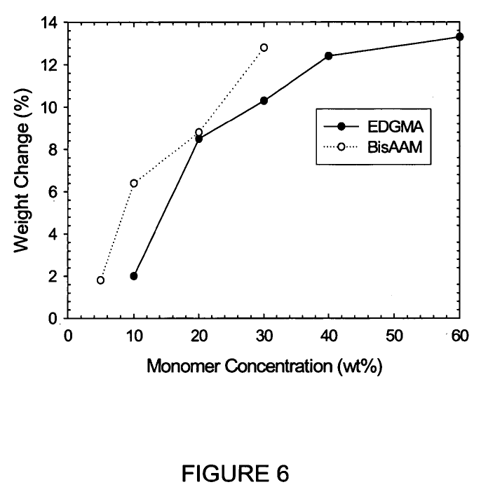

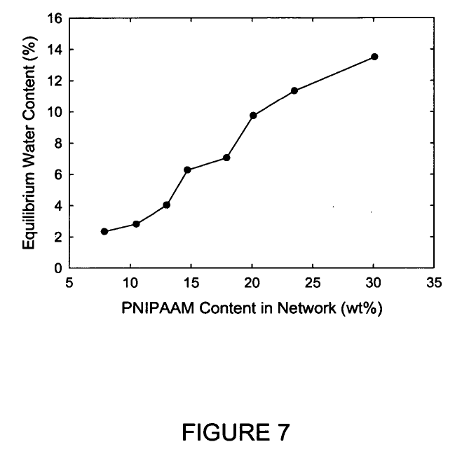

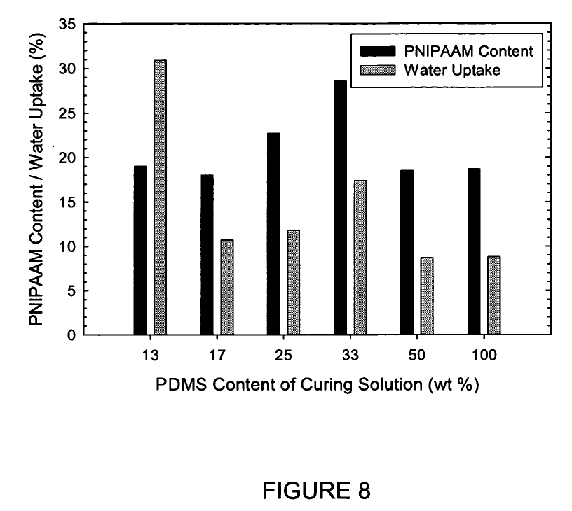

Glucose permeability of the PDMS-PNIPAAM network polymers was expected to be highly dependent on the PNIPAAM content of the polymers and also on the size of the PNIPAAM domains. Therefore, in order to optimize the PNIPAAM content of the membrane as well as to control the size of the PNIPAAM domains, several reaction parameters, including functionality of the PDMS prepolymers, and effect of the addition of solvent during the NIPAAM incorporation step as well as the effect of PNIPAAM crosslinker and initiator concentration were examined. The PNIPAAM content of the resultant networks was found to be relatively unaffected by the concentrations of either the crosslinker or initiator (results not shown). There was a small effect on the PNIPAAM content of the network when ethylene glycol dimethacrylate (EDGMA) was used as a crosslinking agent compared with bis acrylamide, particularly at high crosslinker concentrations as shown in FIG. 6 (which shows a comparison of the different crosslinkers effect, the corsslinker concentrations were 2.0% mol of monomer. The reactions were performed at 4 hours socking time and with the distance to UV lamp 4 cm.) However, within a reasonable range of concentrations, the effect of the crosslinker on the networks was relatively small. The effect of the PDMS as well as the nature of the solvent used during PDMS curing on the resultant network properties was however significantly greater. Water uptake of the PDMS networks prepared from vinyl terminated prepolymer, shown in FIG. 7, clearly demonstrates that water uptake increases with the PNIPAAM content of the network. In this case, the amount of PNIPAAM in the network was controlled by the swelling in the NIPAAM monomer. A similar trend of water uptake results were obtained for the networks prepared using hydroxy terminated prepolymers. However the water uptake of hydroxyl terminated PDMS-PNIPAAM networks can achieve as high as 35%, Curing in solvent was found to result in membranes with similar PNIPAAM contents, but, depending on the fabrication conditions, dramatically higher water uptakes as shown in FIG. 8. FIG. 8 shows the effect of solvent content in PDMS curing solution on the PNIPAAM content of the resultant IPN and on the water uptake of the IPN. All the IPNs were synthesized at same condition expect the PDMS curing solution concentration. Note the parallel trend of PNIPAAM content and water up take in general. However, PDMS control in curing at 13% had highest swelling ratio based on its IPN weight, likely due to much looser PDMS network formed in that case.

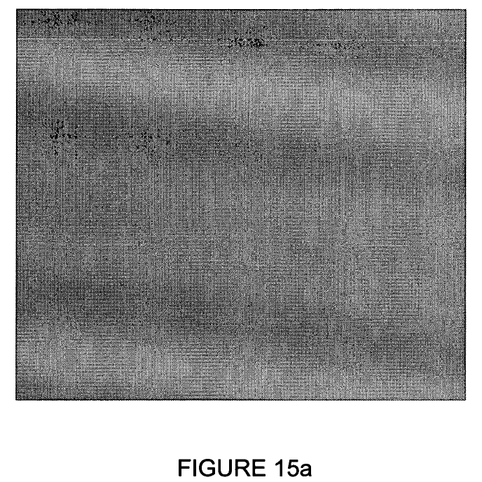

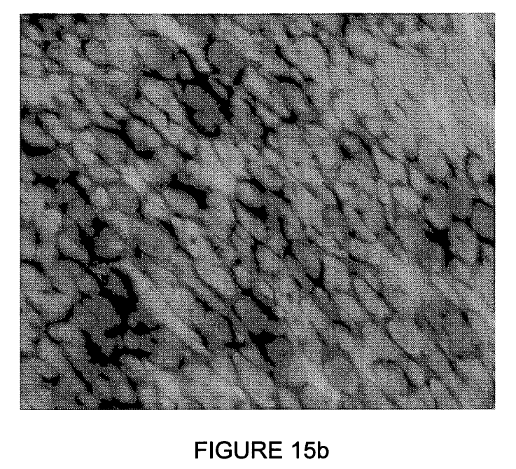

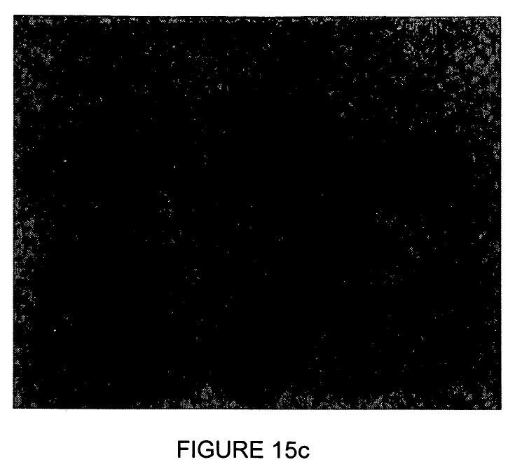

This is believed to be due to the PDMS-OH host polymers prepared in this way having a more open structure allowing for the formation of larger PNIPAAM domains within the PDMS network. TEM images of the networks in FIGS. 15a to 15c support this view. The image of PDMS control shown in FIG. 15a presents only one phase while in contrast the images of hydroxyl and vinyl terminated PDMS-PNIPAAM networks shown in FIG. 15b, 15c clearly show two phases. The morphology of hydroxyl terminated PDMS-PNIPAAM networks is more open and more continuous than that of vinyl terminated PDMS-PNIPAAM networks. Its domain size demonstrated in FIG. 15b is significantly larger comparing with vinyl terminated PDMS-PNIPAAM networks. The connectivity of the PNIPAAM phase of networks provide the material with water swelling and glucose permeation channel. On the other hand, the continuous phase of PDMS performs structure and channel for oxygen permeation. PNIPAAM domain sizes in the networks synthesized from PDMS hosts cured in solvent were significantly larger than those observed in networks synthesized from “neat” cured PDMS. Furthermore, the effect of solvent curing was considerably greater than the effect of for example, increasing the molecular weight of the PDMS prepolymers, which should lead to more open networks and higher molecular weights between crosslinks.

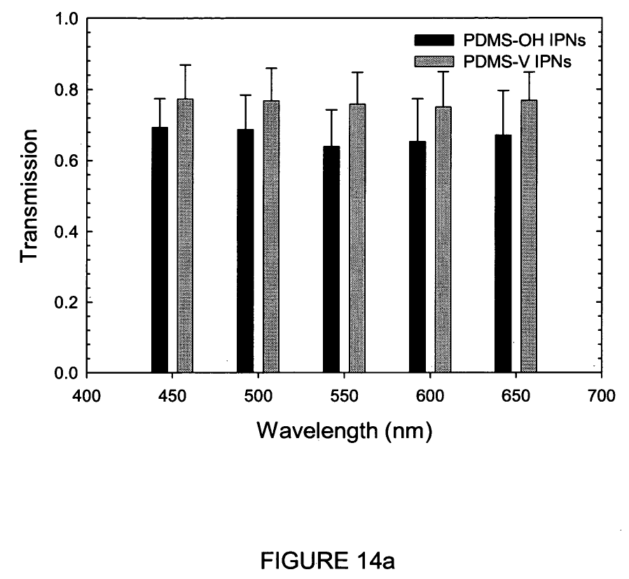

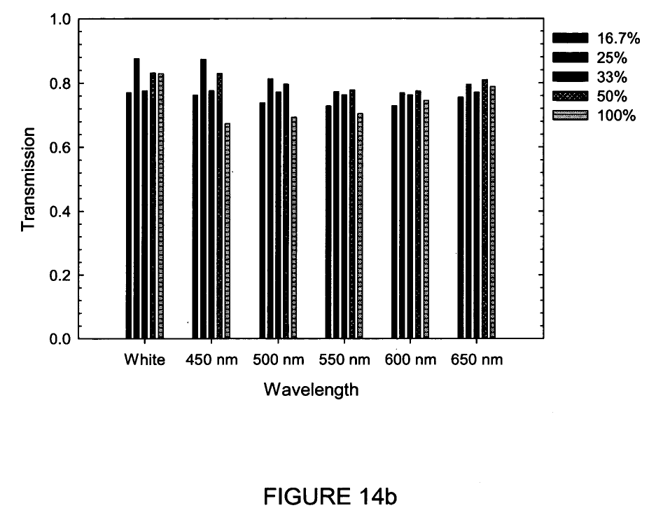

Transparency measurements, summarized in FIG. 14a demonstrate that, depending on the composition of the networks and on the synthesis procedure, membranes with transparency similar to that of the native cornea or commercially available contact lenses could be obtained. Generally vinyl-terminated PDMS prepolymers resulted in the formation of more transparent membranes. The addition of solvent during PDMS curing clearly did not adversely impact the transparency of the membrane as shown in FIG. 14b.

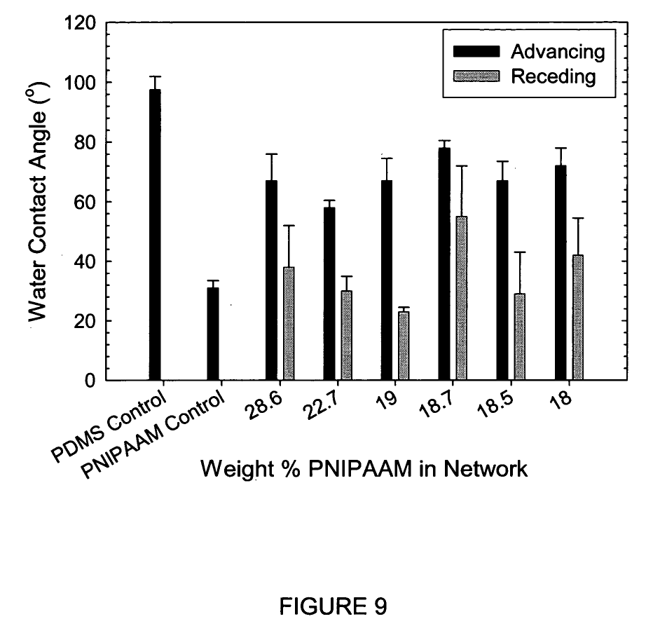

Somewhat surprisingly, the surface properties of the networks were also significantly altered by network formation. The water contact angles measured on the various network surfaces, summarized in FIG. 9, clearly demonstrate the presence of the PNIPAAM on the surface of the polymers. While there were differences in the water contact angles, there were no apparent trends, either as a function of the PNIPAAM content of the membranes or as a function of the water uptake, which may provide a more accurate assessment of the accessibility of the PNIPAAM surface domains to water. The water contact angles measured on the membrane surface exposed to water during the preparation phase were slightly lower than those measured on the membrane surface exposed to air. However, these differences again were not significant.

Surface chemistry, as determined by XPS, of the PDMS surface and a network containing 19.5% PNIPAAM is summarized in Table 1. There are clear differences between the different surfaces and there is evidence to suggest that PNIPAAM is present at the interface, particularly the presence of a significant N1s signal on the network surfaces even in the high vacuum environment of the XPS. The higher C1s levels on the surfaces measured at a takeoff angle of 20° is likely indicative of atmospheric contamination rather than surface enrichment of PDMS since the Si2p levels remained relatively constant in all cases.

Interestingly, the N1 s signal is actually higher at a takeoff angle of 90° than at a more surface sensitive 20°. However, this is more likely the result of reorientation of the PDMS in the high vacuum XPS environment than real differences in the surfaces. Differences were again noted between the surface prepared with exposure to air and the surface prepared with exposure to water.

TABLE 1
Summary of XPS results

| | Takeoff | | | | |
|---|---|---|---|---|---|
| Membrane | Angle (°) | C1s | O1s | N1s | Si2p |
| PDMS-OH control | 90 | 40.9 | 27.8 | 0.8 | 22 |
| | 20 | 50.3 | 25.6 | 0.6 | 23.5 |
| PDMS-OH PNIPAAM | 90 | 53.8 | 24.3 | 2.4 | 19.5 |
| (19.5%) IPN-Water | 20 | 54.9 | 23.9 | 1.7 | 19.5 |
| side | | | | | |
| PDMS-OH PNIPAAM | 90 | 52.7 | 24.9 | 2.0 | 20.4 |
| (19.5%) IPN-Air side | 20 | 54.0 | 24.7 | 0.6 | 20.7 |

TABLE 2
Glucose Permeation Results

| | Solvent Used | PNIPAAM | | |
|---|---|---|---|---|
| | in PDMS | Content in | Water Uptake | Permeability |
| PDMS Host | Cure (wt %) | IPN (wt %) | of IPN (%) | (cm²/s) |
| PDMS-vinyl | 0 | 0 | 0 | ˜0 |
| PDMS-vinyl | 0 | 45.5 | | ˜0 |
| PDMS-OH | 0 | 28.1 | 21.2 | ˜0 |
| PDMS-OH | 66 | 50.9 | 30.7 | 2 × 10⁻¹² |
| PDMS-OH | 87.5 | 21.8 | 30.9 | 7 × 10⁻⁷ |

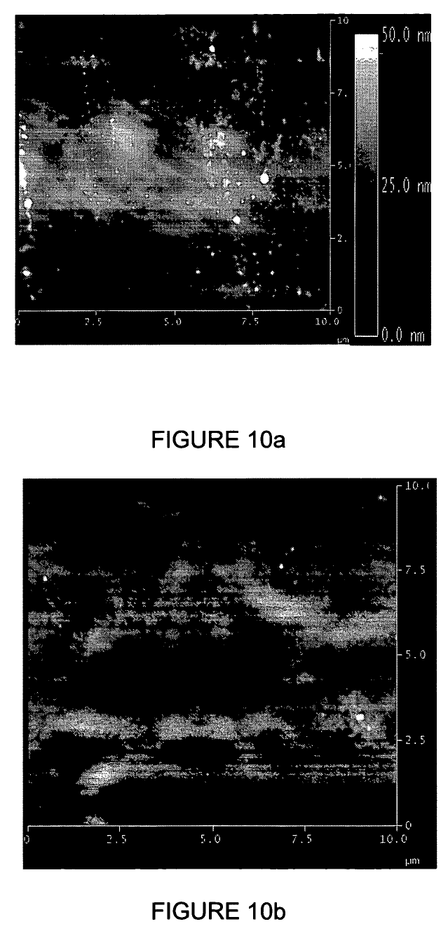

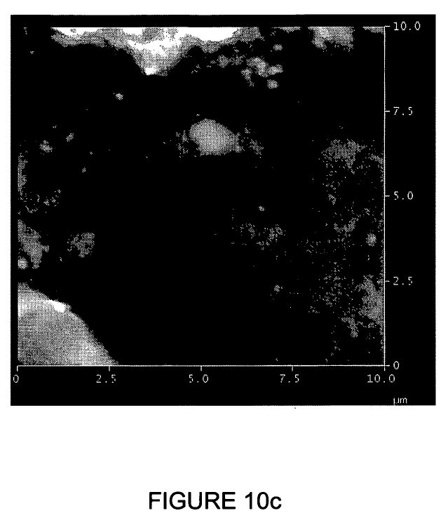

Typical Atomic Force Microscopy images for the pure PDMS host polymers, the vinyl terminated PDMS/PNIPAAM networks and the hydroxy terminated PDMS/PNIPAAM networks are shown in FIGS. 10a and 10b. As shown in FIGS. 10a and 10b, the PDMS control surfaces are very smooth. After the introduction of PNIPAAM into PDMS matrix, the surface shown in FIG. 10c becomes considerably rough. This is attributed to the presence of PNIPAAM on the surface of the network material.

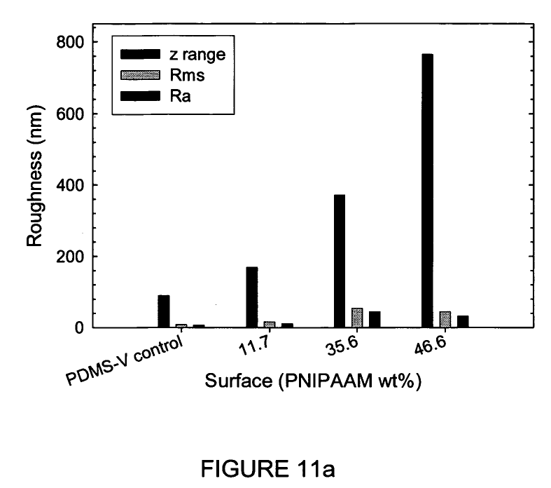

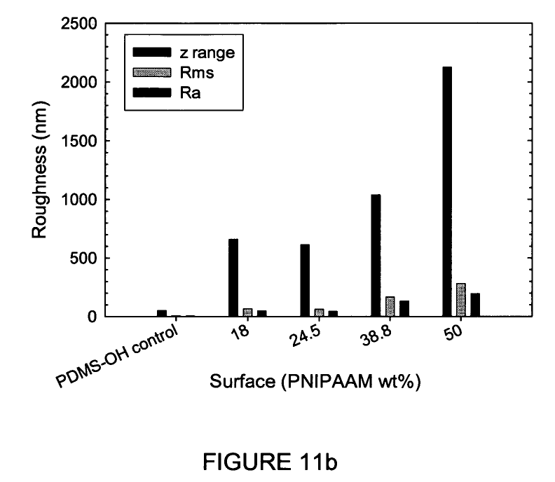

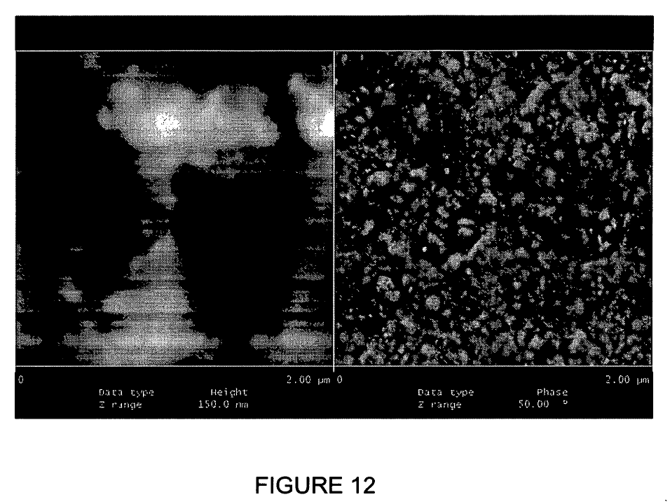

The presence of the PNIPAAM on the surface is evident, particularly for the hydroxy terminated networks. Roughness analysis of the membranes is shown in FIGS. 11a and 11b. It can be seen that the roughness of the polymers increases with increasing PNIPAAM content. Furthermore, the hydroxy terminated PDMS network surfaces shown in FIG. 11b show significantly greater roughness than the vinyl terminated networks shown in FIG. 11a. This is thought to be due to the presence of greater amounts of PNIPAAM at the interface in these polymers which is necessary for significant glucose permeation to occur. Consistent with the water contact angles and the XPS results obtained for the two sides of the membranes, there was a difference in the roughness between the side of the membrane exposed to air during preparation and the side exposed to water, with the water side being significantly rougher. This roughness is thought to be the result of the presence of PNIPAAM domains at the interface. A phase contrast image of the PDMS-OH-PNIPAAM polymer, shown in FIG. 12 provides further evidence for the presence of two phases at the interface.

Taken together, the bulk characterization results, which clearly demonstrate that PDMS hydrogel network polymers have been produced and the surface characterization results which provide evidence for the presence of significant hydrogel at the interface, suggest that these novel network polymers, with both hydrophobic and hydrophilic components are potentially useful in biomedical and drug delivery applications. In ophthalmic applications, glucose permeation is essential for ocular health. Glucose permeation results for the various networks are summarized in Table 2. While PDMS is essentially glucose insoluble, depending on the fabrication method and the PNIPAAM content, it was possible to synthesize networks with significant glucose permeability, comparable to that of the native cornea. Glucose permeability of the networks is expected to depend on a number of factors including the presence of PNIPAAM at the surface, the connectivity of the PNIPAAM in the host PDMS polymer and the size of the PNIPAAM domains. While the amount of PNIPAAM in the polymer network does affect some of these parameters, it cannot be used as the only measure of glucose permeability. PDMS/PNIPAAM polymer networks cast neat could be formed to contain as much as 45% PNIPAAM. However, the host PDMS in this case is expected to have a much tighter structure and therefore the domain size of the resulting networks is significantly smaller. As expected, these membranes were essentially impermeable to glucose, similar to the PDMS. However, when the PDMS was cured in the presence of the solvent, resulting in the formation of a more open network and therefore larger PNIPAAM domains, permeabilities on the order of 10⁻⁷ cm²/s were achievable. These results compare with that for the native cornea which is estimated based on literature results to be between 10⁻⁶ and 10⁻⁷ cm²/s [29]. However, again it should be noted that neither the hydrogel (PNIPAAM) content of the polymer nor the water uptake could be used to predict the glucose permeability, which is expected to depend on a variety of factors.

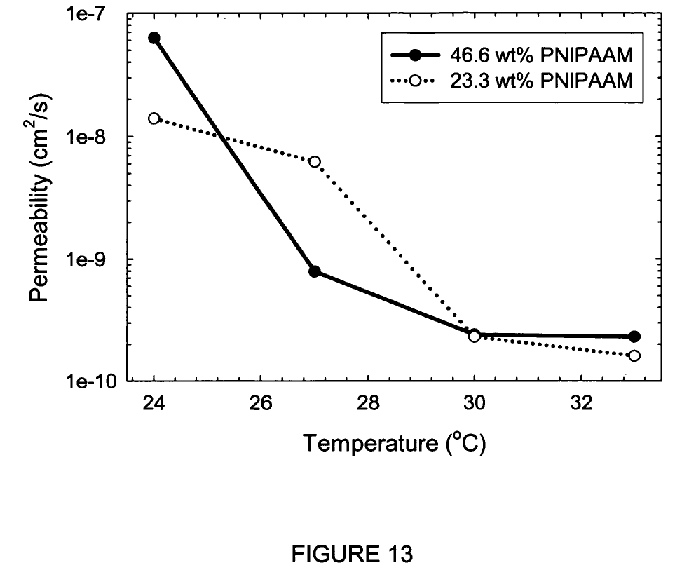

While the very low glucose permeability was unexpected for the membranes containing 50%+PNIPAAM, it seems likely that the surface and or bulk connectivity of these membranes is not adequate for significant transport of glucose through the membranes. Furthermore, the order of magnitude or greater decrease in glucose permeability with increasing permeation temperature above the LCST of the polymers, as shown in FIG. 13 provides additional evidence that the glucose transport in these membranes is determined by the size and nature of the PNIPAAM phase. However it is interesting to note that that a measurable glucose permeability remains in these polymers. Furthermore, it appears that at higher temperatures, particularly above the LCST, the glucose permeability is less dependent on the PNIPAAM content of the polymers.

It will be understood that the present invention has been exemplified using the combination PDMS-PNIPAAM system. However, it is understood that other combinations of materials are contemplated to work. The hydrogel provides the increased wettbility of surfaces and the water channels created by PNIPAAM in the matrix is attribute to the glucose permeation. In this material, preferable PNIPAAM also enhance the mechanical property. The interpenetrating polymer matrix biomaterial composition may be produced where the hydrogel is a poly N-alkyl acrylamide or poly N, N dialkyl acrylamide. The poly N-alkyl acrylamide may be poly (N-isopropyl acrylamide) (PNIPAAM) or the polymers of acrylamide (H2C═CHCONH2) and N-cyclopropylacrylamide N-n-propylacrylamide, N-ethylacrylamide, N-tert-butylacrylamide. The preferred poly dialkylsiloxane may be poly(dimethyl siloxane) (PDMS) or poly diethylsiloxane, ethylhydeosiloxane, methylhydrosiloxane or their block copolymers. Also the carbinol functional methylsioxane, block copolymers carbinol(hydroxyl) terminated PDMS with PEO could be used for the preparation.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims.

TABLE A

| | Water uptake of samples at different temperatures % | | | | | | | | |
|---|---|---|---|---|---|---|---|---|---|
| Sample | 24° C. | 28° C. | 33° C. | 36° C. | 40° C. | 44° C. | 50° C. | 55° C. | 60° C. |
| 20% AAm, 80% | 51.7 | 51.8 | 48.2 | 45.5 | 43.4 | 40.0 | 35.2 | 30.0 | 27.5 |
| NIPAAM | | | | | | | | | |
| 30% AAc, 70% NIPAAM | 30.5 | 41.0 | 54.3 | 48.1 | 56.4 | 60.8 | 61.2 | 78.0 | 67.3 |

References

[1] Chirila T V, Hicks C R, Dalton P D, Vijayasekaran S, Lou X, Hong Y, Clayton A B, Ziegelaar B W, Fitton J H, Platten S, Crawford G J, Constable I J. Artificial Cornea. Progr Polym Sci 1998;23:447-73.

[2] Lopez-Alemany A, Compan V, Refojo M F. Porous structure of Purevisiont versus Focuss Night and DayTM and conventional hydrogel contact lenses. J Biomed Mater Res (Appl Biomater) 2002;63:319-25.

[3] Lai Y C, Valint Jr. P L. Control of properties of silicone hydrogels using a pair of hydrophilic monomers. J Appl Polym Sci 1996;61 :2051-8.

[4] Lai Y C, Friends G D. Surface wettability enhancement of silicone hydrogel lenses by processing with polar plastic molds. J Biomed Mater Res 1997;35:349-56.

[5] Hsuie G. H., Lee S. D., Wang C. C., Shiue M. H. I., and Chang P. C. T., Plasma-induced induced graft copolymerization of HEMA on to silicone rubber and TPX film improving rabbit corneal epithelial cell attachment and growth, Biomaterials 15:163-171 (1994).

[6] Aucoin L, Grif.th C M, Pleizier G, Deslandes Y, Sheardown H. Interactions of corneal epithelial cells and surfaces modi.ed with cell adhesion peptide combinations. J Biomater Sci Polym Edn 2002;13:447-62.

[7] Merrett K, Grif.th M, Dub!e M A, Sheardown H. Interactions of corneal cells with TGF-b2 modi.ed poly dimethyl siloxane surfaces. J Biomed Mater Res 2003;67A:981-93.

[8] Zhang X Z, Yang Y Y, Chung T S. Effect of mixed solvents on characteristics of poly (N-isopropylacrylamide gels). Langmuir 2002;18:2538-42.

[9] Lin Chih-Hsiang et al., Thermal responsive water soluble polymers, U.S. patent application Ser. No. 20030120028

[10] Yoshioka H, Mikami M., Mori Y., Preparation of poly(N-isopropylacrylamide)-b-poly(ethylene glycol) and calorimetric analysis of its aqueous solution, J. M. S.—Pure Appl. Chem., A31(1): 109-112, 1994

[11] Hsuie G H, Hsu S, Yang C C, Lee S H, Yang I K. Preparation of controlled release ophthalmic drops for glaucoma therapy using thermosensitive poly N-isopropylacrylamide. Biomaterials 2002;23:457-62.

[12] Sperling L H, Mishra V. In: Kim S C, Sperling L H, editors. IPN's around the world. New York: Wiley; 1997.

[13] Sperling L H. Polymeric multicomponent materials: an introduction. New York: Wiley; 1997.

[14] Hron P, Slechtova J, Smetana K, Dvorankova B, Lopour P. Silicone rubber hydrogel composites as polymeric biomaterials 0.9.Composites containing powdery polyacrylamide hydrogel. Biomaterials 1997;18:1069-73.

[15] Hron P, Slechtova J. Shape memory of composites based on silicone rubber and polyacrylamide hydrogel. Angew Makromol Chem 1999;268:29-35.

[16] Khorasani M T, Mirzadeh H, Sammes P G. Laser surface modi.cation of polymers to improve biocompatibility: HEMA grafted PDMS, in vitro assay—III. Rad Phys Chem 1999; 55: 685-9.

[17] Abbasi F, Mirzadeh H, Katbab M. Sequential interpenetrating networks of poly (2-hydroxyethyl methacrylate) and polydimethylsiloxane. J Appl Polym Sci 2002;85:1825-31.

[18] Abbasi F, Mirzadeh H, Katbab M. Comparison of viscoelastic properties of polydimethylsiloxane/poly(2-hydroxyethyl methacrylate) IPNs with their physical blends. J Appl Polym Sci 2002; 86:3480-5.

[19] Abbasi F, Mirzadeh H. Properties of poly(dimethylsiloxane)/hydrogel multicomponent systems. J Polym Sci B: Polym Phys 2003;41:2145-56.

[20] Shin M S, Kim S J, Kim I Y, Kim N G, Song C G, Kim S I. Swollen behaviour of crosslinked network hydrogels based on poly (vinyl alcohol) and poly dimethyl siloxane. J Appl Polym Sci 2002;85: 957-64.

[21] Kim S J, Shin M S. Synthesis and characteristics of inter penetating polymer network hydrogels based on silicone and poly(vinyl alcohol). Funct Biomat Key Eng Mater 2001 ;198: 301-7.

[22] Turner J S. Cheng Y L Morphology of PDMS-PMM IPN membranes. Macromolecules 2003;36:1962-6.

[23] Turner J S, Cheng Y L. Preparation of PDMS-PMAA interpenetrating polymer network membranes using the monomer immersion method. Macromolecules 2000;33:3714-8.

[24] Muniz E C, Geuskens G. Polyacrylamide hydrogels and semiinterpenetrating networks with poly (N-isopropylacrylamide): mechanical properties by measure of compressive elastic modulus. J Mater Sci Mater Med 2001;12:879-81.

[25] Kim S J, Park S J, Kim S I. Synthesis and characteristics of interpenetrating polymer network hydrogels composed of poly (vinyl alcohol) and poly (N-isopropylacrylamide). React Funct Polym 2003;55:61-7.

[26] Zhang J, Peppas N A. Synthesis and characteristics of pH and temperature sensitive poly (methacrylic acid)/poly (N-isopropylacrylamide) interpenetrating polymer networks. Macromolecules 2000;33:102-7.

[27] Zhang J, Peppas N A. Morphology of poly (methacrylic acid)/poly (N-isopropyl acrylamide) interpenetrating polymer networks. J Biomater Sci Polym Edn 2002;13:511-25.

[28] Stile R A, Healy K E. Poly (N-isopropylacrylamide)-based semiinterpenetrating polymer networks for tissue engineering applications. 1. effects of linear poly (acrylic acid) chains on phase behaviour. Biomacromolecules 2002;3:591-600.

[29] McCarey B E, Schmidt F H. Modeling glucose distribution in the cornea. Curr Eye Res 1990;9:1025-39.

[30] Kunzler J. F., Salamone T. C., Contact lens material technology, Abstract of paper, 225th ACS National Meeting New Orleans La. United State, Mar. 23-27, 2003 (2003) Poly-029

Therefore what is claimed is:

1. A process for the manufacture of an interpenetrating polymer network biomaterial matrix composition comprising at least a first polymer material and a second polymer material in intimate entanglement one with the other; said process comprising:
a) vulcanizing to effect cross-linking of a vulcanisable silicone polymer backbone precursor of the general formula (I)
T-Qₙ-T (I)
wherein n is greater than or equal to O;
Q is an internal siloxane group of the formula (II)
—O—Si(R¹R²)— (II)
wherein R¹, R² may be the same or different and selected from the group consisting of H, provided that both R¹, R² are not hydrogen on the same internal silicon atom; alkoxy, alkyl, aryl, functional aryl, a crosslinked organic group linking to another silicone-based chain, or a group having an internal siloxane group of the formula III:
(OSiR³R⁴)ₘR⁵ (III)
wherein m is ≧0; and R³, R⁴ and R⁵ for each internal siloxane group may be the same or different selected from the group consisting of alkoxy, siloxy, alkyl, functional alkyl, aryl, functional aryl, independently H, with the proviso that not more than one of R³, R⁴ and R⁵ is H on the same silicon atom; or a crosslinked organic group linking to another silicone-based chain;
T is a radical of the formula (IV);
—OSiR⁶R⁷R⁸ (IV)
wherein R⁶ R⁷ R⁸ may be the same or different and selected from the group consisting of H with the proviso that the silicon atom has no more than one H; alkoxy, siloxy, alkyl, functional alkyl, aryl, functional aryl, or a crosslinked organic group linking to another silicone-based chain;
in a suitable first solvent with a suitable cross-linking agent, to produce a cross-linked elastomer;
b) removing said solvent to form a film of said elastomer;
c) adding a cross-linkable hydrogel compound in a suitable second solvent to said elastomeric film to effect swelling of said elastomer film and form a swollen admixture;
d) reacting said hydrogel compound in said admixture with a suitable cross-linking agent to produce cross-linked hydrogel in said admixture;
and removing said second solvent to produce said interpenetrating polymer network biomaterial matrix composition;
the improvement wherein said silicone polymer backbone precursor concentration in said first solvent is not greater than 60% W/V in said vulcanization step.

2. A process as defined in claim 1 wherein said first silicone polymer backbone precursor concentration is selected from between 5-30% W/V.

3. A process as defined in claim 1 wherein said precursor is a polydimethyl siloxane (PDMS).

4. A process as defined in claim 1 wherein said precursor is a hydroxyl terminated polydimethyl siloxane (PDMS) having an approximate M.W of about 60,000 and a viscosity of approximately 5,000 centistokes.

5. A process as defined in claim 1 wherein said hydrogel is a poly N-isopropyl acrylamide (PNIPAAM).

6. A process as defined in claim 5 including curing said PDMS in an effective solvent for controlling porosity, swellability, and to allow for the incorporation of larger amounts of PNIPAAM during the subsequent interpenetrating network formation.

7. A process as defined in claim 5 including copolymerization of the NIPAAM during matrix formation with varying amounts of acrylamide (AAm) or acrylic acid (AAc).

8. A process as defined in claim 1 wherein said first solvent is toluene.

9. A process as defined in claim 1 wherein said cross-linking agent is tetramethylorthosilicate.

10. A process as defined in claim 1 wherein said second solvent is tetrahydrofuran.

11. An interpenetrating polymer matrix biomaterial composition comprising at least a first polymer and a second polymer in intimate entanglement one with the other, when made by a process as defined in claim 1.

12. A membrane formed of said biomaterial matrix composition as defined in claim 11.

13. A membrane as defined in claim 12 in the form of an ophthalmic membrane, selected from the group consisting of an artificial cornea and a lens.

14. An interpenetrating polymer matrix biomaterial composition comprising at least a first polymer and a second polymer in intimate entanglement one with the first polymer, said first polymer being a poly dialkylsiloxane and said second polymer being a hydrophilic hydrogel.

15. The interpenetrating polymer matrix biomaterial composition according to claim 14 wherein the hydrophilic hydrogel is a poly N-alkyl acrylamide.

16. The interpenetrating polymer matrix biomaterial composition according to claim 15 wherein the poly N-alkyl acrylamide is one of acrylamide (H₂C═CHCONH₂), N-cyclopropylacrylamide or N,N-dialkyl-substituted polyacrylamide.

17. The interpenetrating polymer matrix biomaterial composition according to claim 15 wherein the poly N-alkyl acrylamide is poly (N-isopropyl acrylamide) (PNIPAAM).

18. The interpenetrating polymer matrix biomaterial composition according to claim 17 wherein the hydrophilic hydrogel is selected from the group consisting of acrylic acid (H₂C═CHCOOH), methacrylic acid (H₂C═C(CH₃)COOH), 1-vinyl-2pyrrolidinone, poly(2-hydroxyethyl methacrylate)(CH₂═CHCOOCH₂CH₂OH) and their copolymers.

19. The interpenetrating polymer matrix biomaterial composition according to claim 14 wherein the said poly dialkylsiloxane is a hydroxyl terminated poly(dimethyl siloxane) (PDMS-OH), and wherein said hydrophilic hydrogel is N-isopropyl acrylamide (PNIPAAM).

20. The interpenetrating polymer matrix biomaterial composition according to claim 14 wherein the said poly dialkylsiloxane is a vinyl terminated poly(dimethyl siloxane) (PDMS-V), and wherein said hydrophilic hydrogel is N-isopropyl acrylamide (PNIPAAM).

21. The interpenetrating polymer matrix biomaterial composition according to claim 14 wherein the hydrophilic hydrogel is selected from the group consisting of poly (2-hydroxyethyl methacrylate) (PHEMA) and poly (N-vinyl pyrrolidone) (PVP).

22. The interpenetrating polymer matrix biomaterial composition according to claims 14 wherein the poly dialkylsiloxane is selected from the group consisting of poly(dimethyl siloxane) (PDMS), poly diethylsiloxane, ethylhydeosiloxane, methylhydrosiloxane and their block copolymers.

23. The interpenetrating polymer matrix biomaterial composition according to claim 14 wherein the hydrophilic hydrogel is one of poly N-alkyl acrylamide and poly N,N dialkyl acrylamide.

24. The interpenetrating polymer matrix biomaterial composition according to claim 14 wherein the poly N-alkyl acrylamide is one of poly (N-isopropyl acrylamide) (PNIPAAM), polymers of acrylamide (H₂C═CHCONH₂) and N-cyclopropylacrylamide N-n-propylacrylamide, N-ethylacrylamide, N-tert-butylacrylamide.

25. A membrane formed of said biomaterial matrix composition as defined in claim 14.

26. A membrane formed of said biomaterial matrix composition as defined in claim 14 in the form of an ophthalmic membrane, selected from an artificial cornea or a lens.
